(19) United States
(12) Patent Application Publication
(10) Pub. No.: US 2022/0145376 A1
(43) Pub. Date: May 12, 2022

(54) METHOD FOR DETECTING EXTRACELLUAR VESICLES

(71) Applicant: SHIMADZU CORPORATION, Kyoto-shi, Kyoto (JP)

(72) Inventors: Makoto WATANABE, Kyoto (JP); Hirotaka FUJIMOTO, Kyoto (JP); Taka-Aki SATO, Kyoto (JP)

(73) Assignee: SHIMADZU CORPORATION, Kyoto-shi, Kyoto (JP)

(21) Appl. No.: 17/611,603

(22) PCT Filed: May 13, 2020
(86) PCT No.: PCT/JP2020/019158

§ 371 Date: Nov. 16, 2021

(30) Foreign Application Priority Data
May 17, 2019 (JP) 2019-093686

(51) Int. Cl. C12Q 1/6818; G01N 27/62

(52) U.S. Cl. CPC C12Q 1/6818; G01N 27/62

(57) ABSTRACT

The present invention provides a method for quantifying a plurality of surface antigens simultaneously, thereby identifying an extracellular vesicle such as an exosome, and detecting and quantifying any surface antigen. Specifically, the present invention provides a method for detecting an extracellular vesicle, the method including: a step of labeling an extracellular vesicle in a sample using a first metal labeling reagent for labeling a nucleic acid and a second metal labeling reagent for labeling an extracellular vesicle surface antigen; and a step of identifying the first and second metal labeling reagents by mass spectrometry.

### TECHNICAL FIELD

The present invention relates to a method for detecting an extracellular vesicle using mass spectrometry. More specifically, the present invention relates to a method for directly detecting an extracellular vesicle by mass spectrometry, and a method for quantifying a surface antigen of the extracellular vesicle.

### BACKGROUND ART

The extracellular vesicle refers collectively to a vesicle released from a cell, and is classified into an exosome, a microvesicle, an apoptotic body, and the like depending on its origin and characteristics. It has been reported that the apoptotic body is released from an apoptotic cell, whereas the exosome and the microvesicle are also released from a healthy cell, although the exosome and the microvesicle are different from each other in origin and size.

In recent years, it has been known that then extracellular vesicle functions in inflammation, immunity, and the like, and may be involved in a disease. In addition, since the extracellular vesicle is contained in a body fluid such as blood, urine, or saliva, a possibility of clinical application of the extracellular vesicle has increased, for example, diagnosis of presence or absence of a disease using the extracellular vesicle as a non-invasive biomarker, development of a therapeutic agent targeting the extracellular vesicle, and a drug delivery system (DDS) using uptake of the extracellular vesicle into a cell.

The extracellular vesicle has a membrane formed of a phospholipid bilayer like a cell, and contains a protein (an enzyme, a cytoskeletal protein, a signal molecule, or the like) and a nucleic acid (DNA, RNA, or the like) therein. A surface of the membrane has a surface antigen that varies depending on an origin of the surface antigen, and for example, in a case of an exosome, presence of surface antigens such as CD9, CD63, and CD81 has been known. Therefore, as in a case of cell analysis, it is possible to analyze an exosome by detecting these surface antigens.

Conventionally, there is a flow cytometer as an analysis method for analyzing a surface antigen of a cell, and it is also possible to perform analysis simultaneously by labeling a plurality of surface antigens with different reagents (Patent Literature 1).

However, since the extracellular vesicle is smaller than a cell, and in particular, the exosome is very small (30 to 200 nm), it has been difficult to directly detect and quantitatively analyze a surface antigen of the extracellular vesicle such as the exosome with a flow cytometer.

When the exosome is analyzed with a flow cytometer, a technique has been used in which it is captured with beads onto which an antibody to an exosome marker is immobilized as a primary antibody, and the captured exosome is detected by a detection reagent (secondary antibody) (Patent Literature 2). As products using this mechanism, MACSPlex Exosome Kit (Miltenyi Biotec), PS Capture™ Exosome Flow Cytometry Kit (FUJIFILM), ExoStep (immunostep), and the like are commercially available.

On the other hand, inductively coupled plasma mass spectrometry (ICP-MS) has been known in which plasma (ICP) is used as an ion source and a generated ion is detected by a mass spectrometry unit (Patent Literature 3). This analysis method is a highly sensitive analysis method capable of simultaneously measuring a plurality of elements qualitatively and quantitatively and also capable of measuring an isotope ratio.

### CITATION LIST

### Patent Literature

### Patent Literature 1: EP 2 356 456 B1

### Patent Literature 2: EP 3 093 664 A1

### Patent Literature 3: WO 2016/109603 A1

### SUMMARY OF INVENTION

### Technical Problem

In order to detect an exosome by a conventional method, it is necessary to capture the exosome with beads onto which at least an antigen-bonding molecule such as an antibody is immobilized, and indirectly detect the exosome with a fluorescently labeled secondary antibody (detection antibody). On the other hand, in order to show that the captured extracellular vesicle is, for example, an exosome, it is necessary to confirm presence or absence of a plurality of exosome surface antigens (CD9, CD63, CD81, and the like), but the conventional method cannot simultaneously quantify the plurality of surface antigens. Therefore, there is a need for a technique for quantifying a plurality of surface antigens simultaneously, thereby identifying an extracellular vesicle such as an exosome, and detecting and quantifying any surface antigen.

Recently, it has become possible to use a plurality of types of antibody staining using a multi-color flow cytometry technique, but this is an optically distinguishing technique. Therefore, a combination of fluorescence corrections is complicated in multi-color analysis of four or more colors due to overlapping of wavelengths, and an inaccurate result is obtained disadvantageously.

### Solution to Problem

As a result of various studies in order to solve the above problems, by separately metal-labeling one or more kinds of surface antigens of an extracellular vesicle contained in a sample and a nucleic acid contained in the extracellular vesicle, then analyzing the sample using mass spectrometry, and simultaneously detecting presence of the surface antigens of the extracellular vesicle and the nucleic acid in the extracellular vesicle, the present inventors have completed a method for detecting an extracellular vesicle from a highly complicated sample. The present inventors have also found that similar detection is possible by separately metal-labeling two or more kinds of different extracellular vesicle surface antigens.

That is, the present invention provides the following.

1. A method for detecting an extracellular vesicle, the method including:

a step of labeling an extracellular vesicle in a sample using a first metal labeling reagent for labeling a nucleic acid and a second metal labeling reagent for labeling an extracellular vesicle surface antigen; and

a step of identifying the first and second metal labeling reagents by mass spectrometry.

2. The method according to the item 1, in which the first metal labeling reagent is an intercalator for DNA labeling.

3. The method according to the item 1 or 2, in which in the step of labeling an extracellular vesicle in the sample, two or more kinds of surface antigens are separately labeled, and identified using two or more kinds of the second metal labeling reagents.

4. A method for detecting an extracellular vesicle, the method including:

a step of labeling an extracellular vesicle in a sample using two or more kinds of metal labeling reagents for separately labeling two or more kinds of different extracellular vesicle surface antigens; and

a step of identifying the two or more kinds of metal labeling reagents by mass spectrometry.

5. The method according to any one of the items 1 to 4, in which the metal labeling reagent for labeling an extracellular vesicle surface antigen is obtained by metal-labeling an antibody to the extracellular vesicle surface antigen.

6. The method according to any one of the items 1 to 5, in which the extracellular vesicle is an exosome.

7. The method according to the item 6, in which the extracellular vesicle surface antigen contains one or two kinds of antigens selected from CD9, CD63, and CD81, or three kinds of antigens of CD9, CD63, and CD81.

8. The method according to any one of the items 1 to 7, in which the sample is whole blood, serum, plasma, urine, saliva, or a spinal fluid derived from a subject, or a cell culture supernatant.

9. The method according to any one of the items 1 to 8, in which the mass spectrometry is inductively coupled plasma mass spectrometry (ICP-MS).

10. The method according to any one of the items 1 to 9, in which

in the step of labeling an extracellular vesicle in the sample, a third metal labeling reagent for detecting a further antigen is used, and

in the step of identifying the metal labeling reagent by mass spectrometry, the third metal labeling reagent is also identified by mass spectrometry.

11. The method according to the item 10, in which the further antigen is selected from a protein, a sugar chain, and a lipid whose expression is increased or decreased in a disease specific manner.

12. A method for determining presence or absence or severity of a disease in a subject, the method including:

a step of detecting an extracellular vesicle by the method according to the item 10 or 11; and

a step of determining presence or absence or severity of a disease in a subject based on the amount of the detected extracellular vesicle.

13. A kit for use in the method according to any one of the items 1 to 3 and 5 to 12, the kit including: a first metal labeling reagent for labeling the nucleic acid; and a second metal labeling reagent for labeling the extracellular vesicle surface antigen.

14. A kit for use in the method according to any one of the items 4 to 12, the kit including two or more kinds of metal labeling reagents for labeling the extracellular vesicle surface antigens.

### Advantageous Effects of Invention

According to the present method, an extracellular vesicle surface antigen is labeled with a metal labeling reagent and measured by mass spectrometry such as ICP-MS, and an extracellular vesicle can be thereby directly detected without capturing the extracellular vesicle by antibody beads and the like. In addition, by a combination with a nucleic acid labeling reagent, it is possible to distinguish whether a detected metal ion amount (expression level of a protein such as an antigen) is derived from metal bonded to an antigen released in a sample or derived from metal bonded to an extracellular vesicle membrane. As a result, it is expected to largely improve detection sensitivity and quantitativity of an extracellular vesicle surface antigen.

### BRIEF DESCRIPTION OF DRAWINGS

FIG. 1 is a graph illustrating, in an exosome fraction prepared from a culture supernatant of a breast cancer cell line MCF7, detection results of ¹⁹¹Ir in a sample (FIG. 1(a)) in which DNA is not labeled with an ¹⁹¹Ir (iridium) labeled intercalator and a sample (FIG. 1(b)) in which DNA is labeled with the ¹⁹¹Ir (iridium) labeled intercalator. The horizontal axis indicates a metal ion intensity of ¹⁹¹Ir (iridium), and the vertical axis indicates the number of events (approximately the number of particles) in which metal ions are detected with the intensity of the horizontal axis.

FIG. 2(a) is a graph illustrating that in an exosome fraction prepared from a culture supernatant of a breast cancer cell line MCF7, a DNA⁺ fraction is 15.32% as confirmed by using labeling with an ¹⁹¹Ir-intercalator as an index. The horizontal axis indicates an ion intensity of ¹⁹¹Ir, and the vertical axis indicates the number of particles. FIG. 2(b) is a graph illustrating that from detection results of CD9 and CD63 in the DNA⁺ fraction of FIG. 2(a), a CD9⁺/CD63⁺ fraction is 82.31%. The horizontal axis indicates an ion intensity of ¹⁷¹Yb (ytterbium) (expression level of CD9), and the vertical axis indicates an ion intensity of ¹⁵⁰Nd (neodymium) (expression level of CD63). FIG. 2(c) is a graph illustrating that from a detection result of CD81 in a DNA⁺/CD9⁺/CD63⁺ fraction, a CD81⁺ fraction is 97.58% (high expression: 39.96%, low expression: 57.62%). The horizontal axis indicates an ion intensity of ¹⁴⁵Nd (expression level of CD81), and the vertical axis indicates the number of particles.

FIG. 3 is a graph illustrating results of staining an ¹⁹¹Ir (iridium) positive fraction (DNA positive fraction) in an exosome fraction prepared from serum with an antibody isotype control. FIGS. 3(a), 3(b), and 3(c) indicate a result of identifying a CD9 positive fraction from an intensity distribution of ¹⁷¹Yb (ytterbium), a result of identifying a CD81 positive fraction from an intensity distribution of ¹⁴⁵Nd (neodymium), and a result of identifying a CD63 positive fraction from an intensity distribution of ¹⁵⁰Nd (neodymium), respectively.

FIG. 4 is a graph confirming an expression distribution of CD9, CD63, and CD81 which are DNA⁺ fractions in an exosome fraction prepared from serum. FIGS. 4(a) and 4(b) indicate a result of identifying a fraction in which CD9 and CD63 are co-expressed (DNA⁺/CD9⁺/CD63⁺ fraction) and a result of identifying a fraction in which CD81 is further expressed (DNA⁺/CD9⁺/CD63⁺/CD81⁺ fraction), respectively.

### DESCRIPTION OF EMBODIMENTS

The present application claims priority to Japanese Patent Application No. 2019 093686 filed on May 17, 2019, the entire contents of which are incorporated herein by reference.

The present invention provides a method for detecting an extracellular vesicle, comprising: labeling an extracellular vesicle in a sample using a first metal labeling reagent for labeling a nucleic acid and a second metal labeling reagent for labeling an extracellular vesicle surface antigen; and identifying the first and second metal labeling reagents by mass spectrometry.

In the present invention, the extracellular vesicle may include, but is not particularly limited to, an exosome, a microvesicle, and an apoptotic body. An extracellular vesicle fraction may generally be isolated and purified from a sample containing these extracellular vesicles using ultracentrifugation or the like. For example, an exosome fraction can be obtained as a precipitate by subjecting a sample containing an exosome to ultracentrifugation at 4° C. and 210,000×G for 43 minutes twice. However, according to the method of the present invention, it is also possible to rapidly detect presence and abundance of an extracellular vesicle without preparing such a fraction, and it is also possible to further detect and isolate an exosome from an exosome fraction obtained by ultracentrifugation.

A nucleic acid to be labeled with the first metal labeling reagent may be DNA or RNA. For example, when DNA is labeled, an intercalator can be used as a labeling reagent. When RNA is labeled, for example, a FastTag reagent (universal linker containing a disulfide group) can be used as a labeling reagent. In this case, a reagent in which a metal ion is bonded to a thiol group of a disulfide of the FastTag reagent may be used.

The first metal labeling reagent may preferably be an intercalator for DNA labeling.

Here, the term “intercalator” refers to a compound that is bonded to DNA and inserted in parallel (intercalated) between DNA base pairs of a double helix. As the intercalator, many compounds each having a planar aromatic ring, including some anticancer agents, have been known, and real time PCR (qPCR) using an intercalator capable of emitting fluorescence has also been generally performed.

The present invention is characterized by using a metal-labeled intercalator. A person skilled in the art can easily understand an intercalator that can be used in the present invention and a method for metal-labeling the intercalator based on the description of the specification and common general knowledge in the art, and can use the intercalator in the method of the present invention. Alternatively, an intercalator that has been metal-labeled in advance is commercially available, for example, from Fluidigm, which can be obtained and used in the present invention.

As the second metal labeling reagent, a reagent obtained by metal-labeling an antibody to an extracellular vesicle surface antigen can be used.

For example, when an extracellular vesicle to be detected is an exosome, examples of a surface antigen thereof may include CD9, CD11a, CD11b, CD11c, CD13, CD31, CD37, CD53, CD63, CD81, CD82, Tsg101, Alix, Gag, Integrins alpha4beta1, ICAM-1, LAMP 1/2, Mac-1, PGRL, MHC-I, MHC-II, HLA-G, AP-1, SNAP, Arp2/3, Annexins, Rab5, Rab7, Rap1B, and RabGD1, and antibodies to these antigens can be suitably used.

When the extracellular vesicle to be detected is a microvesicle, examples of a surface antigen thereof may include integrin, selectin, and CD40, and antibodies to these antigens can be suitably used.

When the extracellular vesicle to be detected is an apoptotic body, examples of a surface antigen thereof may include annexin V and phosphatidylserine, and antibodies to these antigens can be suitably used.

A metal used for labeling may be any atomic species as long as it is a metal, and is not particularly limited. For example, the metal used for labeling can be appropriately selected from lithium (Li), sodium (Na), magnesium (Mg), aluminum (Al), potassium (K), calcium (Ca), scandium (Sc), titanium (Ti), vanadium (V), chromium (Cr), manganese (Mn), iron (Fe), cobalt (Co), nickel (Ni), copper (Cu), zinc (Zn), gallium (Ga), germanium (Ge), rubidium (Rb), strontium (Sr), yttrium (Y), zirconium (Zr), niobium (Nb), molybdenum (Mo), Tc, ruthenium (Ru), rhodium (Rh), palladium (Pd), silver (Ag), cadmium (Cd), indium (In), tin (Sn), antimony (Sb), cesium (Cs), barium (Ba), hafnium (Hf), tantalum (Ta), tungsten (W), rhenium (Re), osmium (Os), iridium (Ir), platinum (Pt), gold (Au), thallium (Tl), lead (Pb), bismuth (Bi), lanthanum (La), cerium (Ce), praseodymium (Pr), neodymium (Nd), samarium (Sm), europium (Eu), gadolinium (Gd), terbium (Tb), dysprosium (Dy), holmium (Ho), erbium (Er), thulium (Tm), ytterbium (Yb), lutetium (Lu), thorium (Th), and uranium (U).

In the present invention, preferably, a metal to be used for labeling may preferably be selected from Ir, Yb, Nd, and the like, for example, because there is little possibility that Ir, Yb, Nd, and the like are present in a natural sample.

In the method of the present invention, one kind of the second metal labeling reagent may be used. Alternatively, two or more kinds of surface antigens can be separately labeled and identified using two or more kinds of the second metal labeling reagents.

For example, when the extracellular vesicle to be detected is an exosome, generally and suitably, one or two kinds of antigens selected from CD9, CD63, and CD81 or three kinds of antigens of CD9, CD63, and CD81 are used as the extracellular vesicle surface antigen.

The present invention also provides, as another embodiment, a method for detecting an extracellular vesicle, comprising:

labeling an extracellular vesicle in a sample using two or more kinds of metal labeling reagents for separately labeling two or more kinds of different extracellular vesicle surface antigens; and

identifying the two or more kinds of metal labeling reagents by mass spectrometry.

In this embodiment, an extracellular vesicle of interest can be detected and quantified without using the first metal labeling reagent for labeling the nucleic acid.

A person skilled in the art can easily understand and perform metal labeling of an extracellular vesicle surface antigen that can be used in the present invention based on the description of the specification and common general knowledge in the art. Alternatively, an antibody to an extracellular vesicle surface antigen that has been metal-labeled in advance is commercially available, for example from Fluidigm, which can be obtained and used in the present invention.

In the method of the present invention, as a sample, a body fluid derived from a subject (whole blood, serum, plasma, urine, saliva, or a spinal fluid) can be used, or a cell culture supernatant can be used when a detection target is a cultured cell.

The method of the present invention can be suitably performed using inductively coupled plasma mass spectrometry (ICP-MS), matrix-assisted laser desorption/ionization (MALDI)-MS, or electrospray ionization (ESI)-MS.

Among these methods, ICP-MS is particularly suitably used in the method of the present invention. ICP-MS has a narrow signal half-value width and a high signal resolution, and therefore makes distinguishment easy and has good quantitativity.

Specific examples of a usable apparatus may include, but are not limited to, CyTOF™ (Fluidigm), MALDI-7090, MALDI-8020, AXIMA Performance, AXIMA Confidence, LC-MS8050, and LC-MS8060 (Shimadzu Corporation).

Specific conditions in mass spectrometry can be appropriately selected according to the number, kinds, and the like of metal labels to be detected as metal ions.

The method of the present invention can identify an extracellular vesicle fraction using a metal labeling reagent for detecting a further antigen, and can simultaneously quantify expression of any biomolecule on an extracellular vesicle surface.

In this case, the further antigen which can be used may not be particularly limited as long as it is localized on an exosome membrane surface, but for example, an antigen selected from a protein, a sugar chain, and a lipid whose expression is increased or decreased in a disease specific manner can be used.

For example, overexpression of a human epidermal growth factor receptor type 2 (HER2) protein has been known in a breast cancer patient (Chemotherapy Research and Practice, Vol. 2012, Article ID 743193, doi: 10.1155/2012/743193), and it has also been reported that HER2 is expressed in an exosome derived from a breast cancer cell.

Therefore, it may be possible to quantify HER2 in an exosome derived from a subject by simultaneously detecting a metal-labeled antibody to HER2 in an exosome fraction derived from a subject identified by the method of the present invention.

In this aspect, the method of the present invention can be used as an aid in diagnosis of presence or absence or severity of a disease in a subject. Specifically, a method for determining presence or absence or severity of a disease in a subject includes: a step of detecting an extracellular vesicle by the method described above; and a step of determining presence or absence or severity of a disease in a subject based on the amount of the detected extracellular vesicle.

Alternatively, it may also be possible to detect expression of a cell surface marker unknown in relation to a disease in an extracellular vesicle by the method of the present invention and to use the expression for analysis of a function of the marker.

Furthermore, conventionally, it has been necessary to use ultracentrifugation or the like for acquiring an extracellular vesicle, but it is expected that the method of the present invention makes it possible to directly detect an extracellular vesicle present in blood and to quantify any surface antigen without performing purification by ultracentrifugation or the like.

The present invention further provides a kit for use in the method of the present invention described above, the kit including: a first metal labeling reagent for labeling a nucleic acid; and a second metal labeling reagent for labeling a extracellular vesicle surface antigen. The present invention also provides a kit for use in the method of the present invention described above, the kit including two or more kinds of metal labeling reagents for labeling extracellular vesicle surface antigens.

Examples of a specific aspect of the method of the present invention may include the following.

A sample containing an exosome may be labeled using a metal-labeled intercalator and a metal-labeled antibody to an exosome surface antigen (CD9, CD63, and CD81), and the labeled sample may be measured by cytometer mass using ICP-MS. First, an extracellular vesicle fraction (DNA⁺ fraction) containing DNA labeled with the intercalator may be identified. Furthermore, an exosome fraction (CD9⁺/CD63⁺/CD81⁺ fraction) can be identified by simultaneously quantifying expression levels of CD9, CD63, and CD81 in the DNA⁺ fraction. That is, the exosome fraction can be detected and identified by identifying the DNA⁺/CD9⁺/CD63⁺/CD81⁺ fraction.

### EXAMPLES

Hereinafter, the present invention will be further described with reference to Examples, but the present invention is not limited to these Examples.

### [Example 1] Detection of Exosome from Cell Culture Supernatant

In this Example, a cell culture supernatant was used as a sample, and an exosome was detected and identified. Specifically, an exosome in a sample was identified by the following steps.

A culture supernatant of a breast cancer cell line MCF7 was subjected to ultracentrifugation at 4° C. and 210,000×G for 43 minutes twice to prepare a precipitate as an exosome fraction. The prepared exosome fraction was resuspended in a PBS solution, and 5.0×10¹⁰ exosome particles in the sample solution were used for DNA labeling and antibody staining. The exosome particles were counted using a nanoparticle tracking assay (Nanosight).

On the other hand, a sample solution containing the same number of particles was separately prepared. A sample in which neither DNA labeling nor antibody staining was performed (negative control 1) and a sample in which only DNA labeling was performed (negative control 2) were also prepared. DNA labeling and antibody staining were performed according to the manufacturer's (Fluidigm) protocol.

Reagents used for antibody staining and DNA labeling of an exosome were as follows.

an intercalator (¹⁹¹Ir-intercalator, Fluidigm),

an anti-CD9 antibody (¹⁷¹Yb labeled anti-CD9 mAb, Fluidigm),

an anti-CD63 antibody (¹⁵⁰Nd labeled anti-CD63 mAb, Fluidigm), and

an anti-CD81 antibody (¹⁴⁵Nd labeled anti-CD81 mAb, Fluidigm)

After antibody staining and DNA labeling, analysis by ICP-MS (CyTOF™, Fluidigm) was performed. DNA in each particle was quantified by quantifying ¹⁹¹Ir ions by ICP-MS. In addition, the expression levels of CD9, CD63, and CD81 were quantified by quantifying the amounts of ¹⁷¹Yb ions, ¹⁵⁰Nd ions, and ¹⁴⁵Nd ions, respectively.

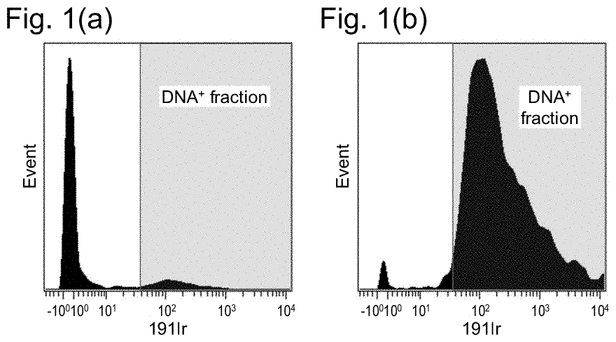

First, a DNA positive fraction (DNA⁺ fraction) was identified from a distribution comparison of the ¹⁹¹Ir ion amount between the negative control 2 (FIG. 1(b)) and the negative control 1 (FIG. 1(a)).

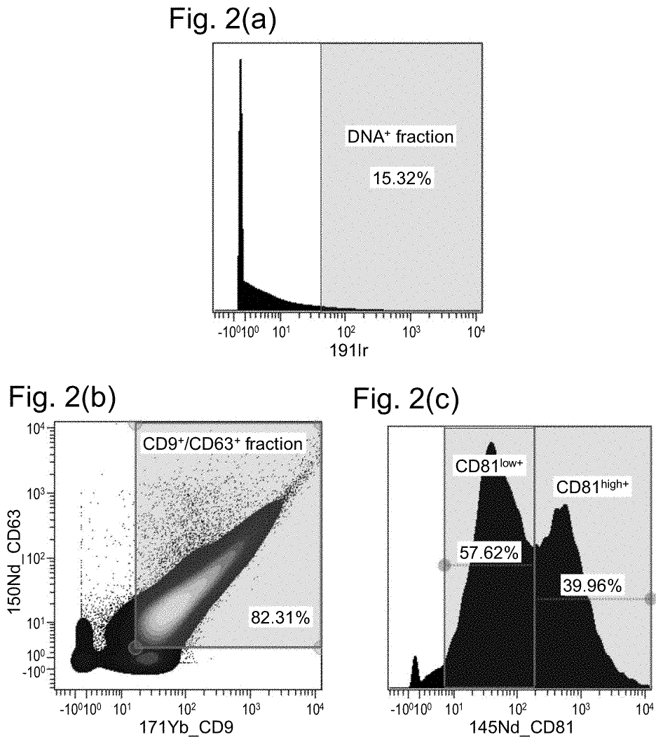

Next, an expression distribution of CD9 and CD63 in an extracellular vesicle in the DNA⁺ fraction (FIG. 2(a)) was confirmed. From this expression distribution, a fraction in which CD9 and CD63 were co-expressed (DNA⁺/CD9⁺/CD63⁺ fraction) was identified (FIG. 2(b)).

Furthermore, an expression distribution of CD81 in the DNA⁺/CD9⁺/CD63⁺ fraction was confirmed, and a fraction in which CD81 was expressed (DNA⁺/CD9⁺/CD63⁺/CD81⁺ fraction) was identified (FIG. 2(c)).

Since this DNA⁺/CD9⁺/CD63⁺/CD81⁺ fraction was a fraction containing DNA and expressing all of CD9, CD63, and CD81 which are exosome markers, this fraction could be confirmed to be an exosome.

### [Example 2] Detection of Exosome from Serum

In this Example, serum was used as a sample, and an exosome was detected and identified. Specifically, according to the steps described in Example 1, an exosome fraction was prepared from human-derived serum by ultracentrifugation. The prepared exosome fraction was resuspended in a PBS solution, and 1.0×10¹⁰ exosome particles in the sample solution were used for DNA labeling and antibody staining.

On the other hand, a sample solution containing the same number of particles was separately prepared. A sample in which only DNA labeling was performed (negative control 1) and a sample stained only with an antibody isotype control (negative control 2) were also prepared. DNA labeling and antibody staining were performed according to the manufacturer's (Fluidigm) protocol.

Reagents used for antibody staining and DNA labeling of an exosome were as follows.

an intercalator (¹⁹¹Ir-intercalator, Fluidigm),

an anti-CD9 antibody (¹⁷¹Yb labeled anti-CD9 mAb, Fluidigm),

an anti-CD63 antibody (¹⁵⁰Nd labeled anti-CD63 mAb, Fluidigm), and

an anti-CD81 antibody (¹⁴⁵Nd labeled anti-CD81 mAb, Fluidigm)

After antibody staining and DNA labeling, analysis by ICP-MS (CyTOF™, Fluidigm) was performed. DNA in each particle was quantified by quantifying ¹⁹¹Ir ions by ICP-MS. In addition, the expression levels of CD9, CD63, and CD81 were quantified by quantifying the amounts of ¹⁷¹Yb ions, ¹⁵⁰Nd ions, and ¹⁴⁵Nd ions, respectively.

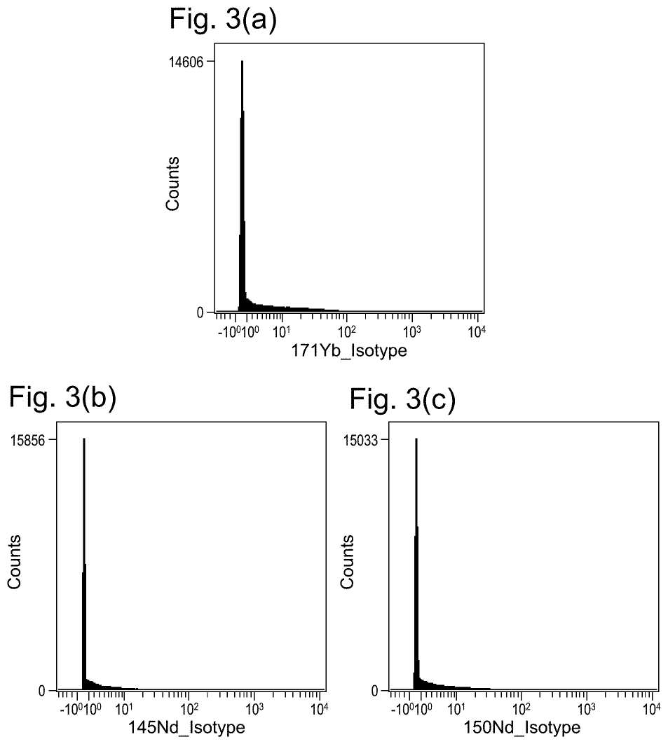

First, an Ir positive fraction, that is, a DNA positive fraction (DNA⁺ fraction) was identified from an ¹⁹¹Ir intensity distribution of the negative control 1. In addition, positive fractions of CD9, CD63, and CD81 were identified from ¹⁷¹Yb, ¹⁵⁰Nd, and ¹⁴⁵ intensity distributions of the negative control 2, respectively (FIG. 3).

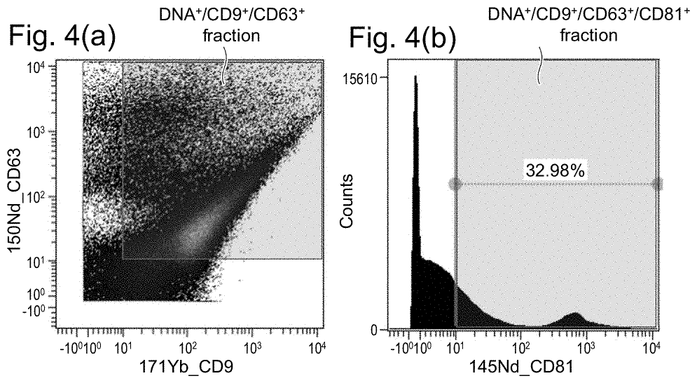

An expression distribution of CD9 and CD63 in an extracellular vesicle of the DNA⁺ fraction in the serum sample was confirmed (FIG. 4(a)). From this expression distribution, a fraction in which CD9 and CD63 were co-expressed (DNA⁺/CD9+/CD63⁺ fraction) was identified (in the upper right frame in FIG. 4(a)). Next, an expression distribution of CD81 in the DNA⁺/CD9⁺/CD63⁺ fraction was confirmed, and a fraction in which CD81 was expressed (DNA⁺/CD9⁺/CD63⁺/CD81⁺ fraction) was identified (FIG. 4(b)). Since this DNA⁺/CD9⁺/CD63⁺/CD81⁺ fraction was a fraction containing DNA and expressing all of CD9, CD63, and CD81 which are exosome markers, this fraction could be confirmed to be an exosome.

From the above, it was confirmed that the analysis method of this Example can also be applied to human serum which is a biological sample, and an exosome can be identified.

### INDUSTRIAL APPLICABILITY

The method of the present invention can be used for diagnosing presence or absence and the degree of a disease in a subject by detecting an expression state of a surface antigen in an extracellular vesicle such as an exosome. The method of the present invention can also be used for detecting expression of a specific surface antigen and examining a function of the antigen.

1. A method for detecting an extracellular vesicle, the method comprising:
a step of labeling an extracellular vesicle in a sample using a first metal labeling reagent for labeling a nucleic acid and a second metal labeling reagent for labeling an extracellular vesicle surface antigen; and
a step of identifying the first and second metal labeling reagents by mass spectrometry.

2. The method according to claim 1, wherein the first metal labeling reagent is an intercalator for DNA labeling.

3. The method according to claim 1, wherein in the step of labeling an extracellular vesicle in the sample, two or more kinds of surface antigens are separately labeled, and identified using two or more kinds of the second metal labeling reagents.

4. A method for detecting an extracellular vesicle, the method comprising:
a step of labeling an extracellular vesicle in a sample using two or more kinds of metal labeling reagents for separately labeling two or more kinds of different extracellular vesicle surface antigens; and
a step of identifying the two or more kinds of metal labeling reagents by mass spectrometry.

5. The method according to claim 1, wherein the metal labeling reagent for labeling an extracellular vesicle surface antigen is obtained by metal-labeling an antibody to the extracellular vesicle surface antigen.

6. The method according to claim 1, wherein the extracellular vesicle is an exosome.

7. The method according to claim 6, wherein the extracellular vesicle surface antigen comprises one or two kinds of antigens selected from CD9, CD63, and CD81, or three kinds of antigens of CD9, CD63, and CD81.

8. The method according to claim 1, wherein the sample is whole blood, serum, plasma, urine, saliva, or a spinal fluid derived from a subject, or a cell culture supernatant.

9. The method according to claim 1, wherein the mass spectrometry is inductively coupled plasma mass spectrometry (ICP-MS).

10. The method according to claim 1, wherein
in the step of labeling an extracellular vesicle in the sample, a third metal labeling reagent for detecting a further antigen is used, and
in the step of identifying the metal labeling reagent by mass spectrometry, the third metal labeling reagent is also identified by mass spectrometry.

11. The method according to claim 10, wherein the further antigen is selected from a protein, a sugar chain, and a lipid whose expression is increased or decreased in a disease specific manner.

12. A method for determining presence or absence or severity of a disease in a subject, the method comprising:
a step of detecting an extracellular vesicle by the method according to claim 10; and
a step of determining presence or absence or severity of a disease in a subject based on the amount of the detected extracellular vesicle.

13. A kit for use in the method according to claim 1, the kit comprising: a first metal labeling reagent for labeling the nucleic acid; and a second metal labeling reagent for labeling the extracellular vesicle surface antigen.

14. A kit for use in the method according to claim 4, the kit comprising two or more kinds of metal labeling reagents for labeling the extracellular vesicle surface antigens.
